(19) United States
(12) Patent Application Publication
(10) Pub. No.: US 2022/0253917 A1
(43) Pub. Date: Aug. 11, 2022

(54) Method for Generating a Shopping and Educational Experience

(71) Applicant: Christian Saxon, Chesapeake, VA (US)

(72) Inventor: Christian Saxon, Chesapeake, VA (US)

(21) Appl. No.: 17/548,734

(22) Filed: Dec. 13, 2021

Related U.S. Application Data
(60) Provisional application No. 63/148,160, filed on Feb. 11, 2021.

(51) Int. Cl. G06Q 30/06; G06Q 30/02

(52) U.S. Cl. CPC G06Q 30/0633; G06Q 30/0605; G06Q 50/205; G06Q 30/0222; G06Q 30/0631; G06Q 30/0617

(57) ABSTRACT

The present disclosure relates to a system and method for generating click packs or packages of gift items selected for a special occasion. The click packs are supplied by a supplier selected from a plurality of suppliers based on bid results. The system and method provides children with a shopping and educational experience allowing them to select gifts, and unlock gifts by clearing, or progressing through, educational programs and problems. Parents can place limits on the cost of the gifts and can share the gift items with other users, thereby allowing the other users to contribute to the gifts. The system and method can use artificial intelligence for creating profiles and recommending gift items to the users.

### CROSS-REFERENCE TO RELATED APPLICATION

The present application claims priority to, and the benefit of, U.S. Provisional Application No. 63/148,160, which was filed on Feb. 11, 2021 and is incorporated herein by reference in its entirety.

### FIELD OF THE INVENTION

The present invention relates generally to the field of online shopping and entertainment. More specifically, the present invention relates to a child's shopping software application and website that allows children and parents to shop together. The child's shopping software application/web site allows the children to set up their profile, and based on their set up profile, the software application packages multiple parameters of the profile and recommends various products of interest for the children. The shopping application of the present invention allows others to buy gifts for children on various occasions, encourages children to save money, allows parents to set shopping thresholds for their children, allows children to play educational games to unlock additional products, etc. Accordingly, the present disclosure makes specific reference thereto. Nonetheless, it is to be appreciated that aspects of the present invention are also equally applicable to other like applications, devices, and methods of manufacture.

### BACKGROUND OF THE INVENTION

By way of background, holiday and birthday shopping can be a stressful endeavor for parents. Usually, children make lists and request certain items of interest for their birthdays or other occasions. Parents go through the list and search for each item in online stores. This is a time-consuming and labor-intensive process. Further, the current shopping methods for kids do not allow much input or interaction from the children themselves. Even the wish lists of children, parents may buy their choice of gifts for their children as per their convenience. Without receiving the desired gifts, children may be left unhappy and unsatisfied.

Additionally, many children are not tech savvy or old enough to use the internet to select items on retail websites, thereby causing parents to search and buy items for children. Currently, there are not kid-friendly websites or applications (apps) to allow children to participate in the shopping process. Children do not gain any knowledge of online shopping and may be unaware of shopping processes.

Further, other individuals such as grandparents, relatives or others may desire to gift something to children on the occasion of birthdays, holidays or the like. With growing age and trending technology, interests and choices of the children can change drastically. Other individuals may not be able to choose the desired item for gifting the children, thereby leaving the children unsatisfied with the gifts.

Therefore, there exists a long felt need in the art for a kid-friendly website that allows the children to give their input in selecting and receiving gifts for their birthdays, festivals, or the like. There is also a long felt need in the art for a kid-friendly website or software application (app) that enables the children to participate in the shopping process. Additionally, there is a long felt need in the art for a website or software application that eliminates the need to search individual items on multiple online shopping platforms. Moreover, there is a long felt need in the art for a shopping platform that recommends items or gifts of a child's choosing and allows parents, grandparents and other individuals to gift desired items to the child on his or her birthday or other day of celebration. Further, there is a long felt need in the art for a shopping website that considers the likes, dislikes, interests and other parameters of children for recommending products for them. Furthermore, there is a long felt need in the art for a kid-friendly shopping website or software application that can be easily used by children who are not tech savvy. Finally, there is a long felt need in the art for a shopping platform that enables children to get involved in the gift-giving process and assists parents in narrowing down choices for each child without having to check multiple websites and stores.

The subject matter disclosed and claimed herein, in one embodiment thereof, comprises a computer-implemented method and a system for allowing children and parents to engage in the shopping experience for special occasions. The method creates a click pack containing all gift items selected for a given special occasion. More specifically, the method includes: generating profiles of users such as of a child and a parent; receiving selection of gift items from the child for a specific occasion; packaging all the selected gift items into a click pack; sending the click pack package to a plurality of registered suppliers; receiving bids from one or more suppliers to supply the package to the child; and, receiving selection of a supplier for supplying the package. The method further includes providing educational programs to the child for allowing the parent to remove or increase the ceiling limit to purchase the click pack based upon the progress of the educational programs.

In this manner, the novel child-friendly website or software application of the present invention accomplishes all of the forgoing objectives, and provides a relatively easy and convenient solution to allow children to get involved in the gift giving process and assists parents and other individuals in gifting desired items to children on birthdays, etc. The child-friendly website or software application of the present invention allows the children to input their likes, dislikes, interests, and more, and ensures children receive gifts of their choice/interest. Additionally, the child-friendly shopping platform supports various features that improves the overall shopping experience for children, parents and others, and saves time and effort for the users.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a computer implemented method of allowing children and parents to engage in the shopping experience for special occasions and to create a package or click pack of all gift items selected for the given special occasion into one unit. The method comprising: generating a child profile and a parent profile, wherein the profiles are created based on the preferences of the corresponding user; receiving a selection of gift items from the child for a specific occasion; packaging all the selected gift items into a package; sending the package to a plurality of registered suppliers; receiving bids from one or more suppliers to supply the package to the child; and, receiving a selection of a supplier for supplying the package. The method further includes setting a predetermined ceiling limit on the cost of gift items for the specified occasion by the parent. The method further includes providing educational programs for completion by the child in order to increase the predetermined ceiling limit based upon the progress of the educational programs.

In a further embodiment of the present invention, a computer-implemented method of allowing children and parents to engage in the shopping experience for special occasions is described. The method includes: selecting of gift items by a child for a special occasion such as a graduations, holidays, birthdays or any other event that may warrant gift-giving; creating a click pack of all the selected gift items as a single unit; calculating cost of the packaged unit; comparing the cost of the unit with a limit set by the parent(s) for the special occasion; and, sending an email to the parent to request an increase in the predetermined limit. The method can further include sending a link to a relative/friend indicating that one or more items are left to purchase, or extra funding is required to purchase the unit package.

The subject matter disclosed and claimed herein, in one embodiment thereof, comprises a children's shopping and educational system. The system is designed to allow children and parents an opportunity to engage in the shopping experience for special occasions such as graduations, holidays, birthdays or any other event that may warrant gift-giving. The system further comprises: a computer-implemented software application configured to be installed in an electronic device; a server with integrated recommendation engine; and, the software application and the server are in communication allowing the software application to provide shopping interfaces and educational programs to a user of the software application. The recommendation engine recommends items for purchase based on the profile of the user wherein the profile includes likes, dislikes, favorite movies, games, characters, gift types, clothes, colors, animals, interests, TV shows and the like.

In yet a further embodiment of the present invention, a computer-implemented method for providing educational levels to unlock gift items of higher value is described. The method includes: selecting one or more gift items by a child; determining that the cost of the selected items is more than a threshold value; providing educational problems to the child; receiving solutions of the educational problems from the child; and, unlocking the gift when the solutions are correct so that the child can receive the selected gift for an upcoming special occasion.

In yet a further embodiment, a method to recommend a customized gift item to a user is disclosed. The method analyzes the likes, dislikes and wish list of the user and creates a customized profile using artificial intelligence (AI), engaging suppliers that supply items based on a user's AI generated profile wherein the suppliers bid against each other to supply the items to the user.

In summary, the application and method of the present invention helps parents save money by buying bundled packs of toys/gifts from suppliers (i.e., they save money by buying a group of products instead of just one, similar to a monthly subscription box service). Likewise, the application and method of the present invention also helps smaller toy/gift makers/suppliers by saving them money on warehousing/stocking shelves by selling in bulk and also gets them more name recognition so that they can compete with larger manufacturers.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a block diagram of a children shopping system combined with an educational program system of the present invention in accordance with the disclosed architecture;

FIG. 2 illustrates a block diagram showing parameters used for recommendation of products for children using the children's shopping system of the present invention in accordance with the disclosed architecture;

FIG. 3 illustrates a flow diagram showing exemplary steps of recommendation and purchase of products for children in the children shopping platform of the present invention in accordance with the disclosed architecture;

FIG. 4 illustrates a block diagram of showing the different features of the children's shopping platform of the present invention in accordance with the disclosed architecture;

FIG. 5 illustrates a flow diagram showing steps of sharing a link with other users using the children shopping platform of the present invention in accordance with the disclosed architecture;

FIG. 6 illustrates a perspective view showing a child accessing the children's shopping platform interface on a portable device in accordance with the disclosed architecture;

FIG. 7 illustrates a perspective view showing sign-in interface of the software application of the children's shopping platform of the present invention in accordance with the disclosed architecture;

FIG. 8 illustrates a perspective view showing different interests displayed by the software application of the present invention in accordance with the disclosed architecture; and

FIG. 9 illustrates a perspective view of a parent and a child shopping together using the shopping and educational platform of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a kid-friendly website that allows the children to give their input in receiving gifts for their birthdays, holidays or the like. There is also a long felt need in the art for a kid-friendly website or software application (app) that enables the children to participate in the shopping process. Additionally, there is a long felt need in the art for a website or software application that eliminates the need to search individual items on multiple online shopping platforms. Moreover, there is a long felt need in the art for a shopping platform that recommends items or gifts of a child's choosing and allows parents, grandparents and other individuals to gift desired items to the child on their birthday or other occasion. Further, there is a long felt need in the art for a shopping website that considers likes, dislikes, interests, and other parameters for recommending products for children. Furthermore, there is a long felt need in the art for a kid-friendly shopping website or software application that can be easily used by children who are not tech savvy. Finally, there is a long felt need in the art for a shopping platform that enables children to get involved in the gift-giving process and assists parents in narrowing down choices for each child without having to check multiple websites and stores.

The present invention, in one exemplary embodiment, is a computer-implemented method of allowing children and parents to engage in the shopping experience for special occasions is described. The method includes: selecting of gift items by a child for a special occasion such as a graduation, holiday, birthday or any other event that may warrant gift-giving; creating a click pack of all the selected gift items as a single unit; calculating the cost of the packaged unit; comparing the cost of the unit with a limit set by the parent(s) for the special occasion; and, sending an email to the parent to request an increase in the predetermined limit. The method can further include sending a link to a relative/friend indicating that one or more items are left to purchase, or extra funding is required to purchase the unit package.

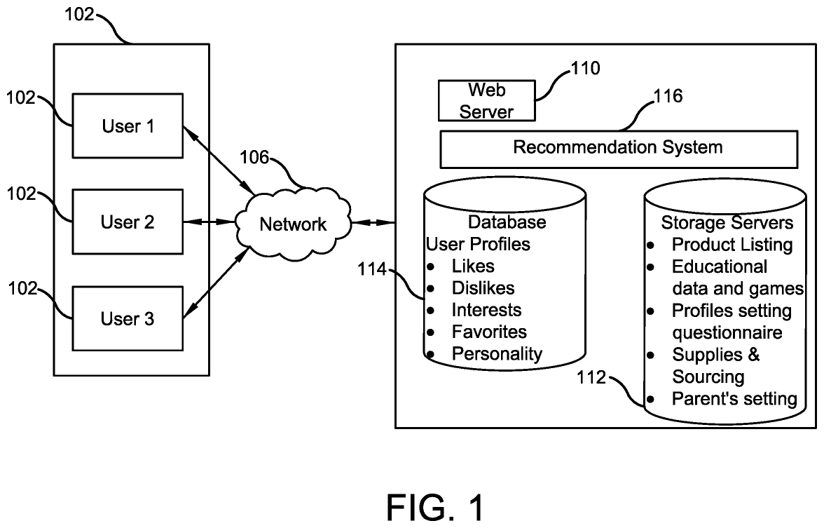

Referring initially to the drawings, FIG. 1 illustrates a block diagram of a children's shopping system combined with an educational program system of the present invention in accordance with the disclosed architecture. Children shopping combined with an educational program system 100 provides children and parents a system to engage in a shopping experience for special occasions such as graduations, holidays, birthdays or any other event that may warrant gift giving.

The children shopping platform system 100 includes a plurality of clients/users 104 operating from a remote location referred to as client site 102. It should be noted that the clients are distributed and can access the system 100 through their portable electronic devices via a software application or a server-hosted website accessed through the internet. The users 104 can access a server site 108 over a network 106 such as, e.g., the Internet. The users 104 can include children, parents, grandparents, siblings, friends, relatives and others who are associated with the subject children and are willing to shop for the children using the shopping platform system 100. Each child or user 104 registers, creates a profile and provides details such as interests, likes, dislikes and other such preferences for enabling product recommendations based on the created user profile. A personalized profile is created in the system 100 for each user 104 that allows artificial intelligence-based product recommendations for the user 104. A user can download a software application from an application such as the Apple App Store or Google Play Store. The downloaded software application provides separate interfaces for the user to create the profile and to do the electronic shopping.

The server site 108 has a server 110 that receives user data over the network 106. The server 110 can be in communication with a user profile database 114 and all the profile data received from the users 104 is stored in the user profile database 114. The server site 108 also provides an interactive website in addition to communication with the software application that allows users such as children 104 to engage in the shopping experience as well as in education levels or modules such as spelling games, math problems or anything that interests the child based on his or her age, grade level, etc. The server site 108 has a storage server 112 that stores data such as product listing, education data and games, profile setting questionnaires, suppliers and sourcing and other such type of data.

It should be understood that all the personal information of the users such as address, contact details, payment details and the like are stored in an encrypted form allowing secure storage of the information. The communication between the clients/users 104 and the server site 108 is also encrypted and any conventional encryption technique can be used for the encrypted communication.

The server site 108 has a recommendation engine 116 which is coupled to the user profile database 114 and the storage server 112. The recommendation engine 116 evaluates the product listing, educational data and games or any other such type of information stored in the storage server 112 and compares this information with the information stored in the user profile database 114. Based on the evaluation and the comparison, the recommendation engine 112 recommends an inventory of products from pre-determined retailers that match the users' 104 profile. The recommendation engine 116 evaluates the users' 104 likes, dislikes or interests and matches the product listing, or educational data & games, on the basis of factors chosen by the user 104 and relevant results are provided to the user 104. The recommendation engine 116 collectively analyzes the data related to the user profile 114 and the storage server 112, and recommends relevant results based on collaborative filtering. The recommendation engine 116 can use state-of-the-art techniques such as machine language and artificial intelligence (AI) for providing recommendations to each user 104.

It should be noted that the recommendation engine 116 can recommend a package of products or a ‘click pack’ for all items for a given special occasion as a single unit. A list of suppliers that are a part of the system 100 can bid for the package to source, supply and send the ‘click packs’. The server 108 sources the requirements of the package to the suppliers with the intention of having the suppliers ‘bid’ on the rights to supply the ‘click packs’ to the user 104.

The system 100 can have an integrated or coupled payment system to allow the users 104 to transfer money from a check card, credit card, an automated clearing house (ACH) transaction via a bank or a one-time or recurring amount from a user account, for shopping any gift items or recommended packages (i.e. click packs) for their children on special occasions such as birthdays, holidays, milestones and more.

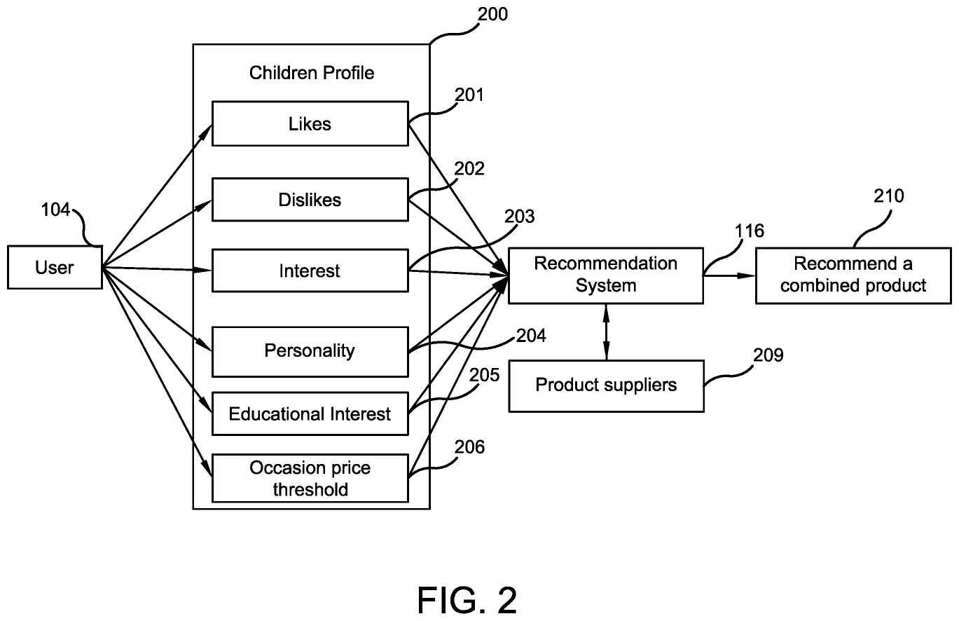

FIG. 2 illustrates a block diagram showing parameters used for recommendation of products for children using the children's shopping system of the present invention in accordance with the disclosed architecture. A user such as a child 104 initially signs-up and creates his/her own profile 200. The profile 200 can be prepared by the child 104 on its own or by the parents of the child. The child profile 200 can be created based on various parameters or by answering questions from a preset questionnaire. The child's response to the preset questionnaire decides the likes 201, dislikes 202, interests 203, personality 204, educational interests 205 and more to create the child's profile 200 for the shopping system 100. Additionally, an occasion price threshold or limit 206 can be separately set by the parents of the child 104 to set a purchase amount limit for the children for any occasion.

The questionnaire may contain a list of colors, animals, toys, objects, items and more to allow the children to input their likes 201 and dislikes 202. To decide the interest parameter 203, the questionnaire can include questions related to favorite movies, games, hobbies and other such types of information. To determine the personality parameter 204 for any child 104, the questionnaire can include questions related to the user's gender, age, product preferences, store preferences and other such types of information. Further, the questionnaire can include questions related to subjects such as science, math or any other such types of subjects to determine the educational interests 205 of the child 104. The educational interest parameter 205 is used for engaging the child 104 in educational levels such as spelling games, math problems, reading comprehension, science or anything that interests the child at the time their initial profile is built.

The questions in the preset questionnaire are not limited, and can be set up as per the requirements. Additionally, the questions can be designed to build the profile 200 of the child 104 using AI technology or other similar technologies known in the state of the art.

In one potential embodiment, once the child 104 completes one or more tasks or modules provided by the educational interest 205 parameter, another or next round of gifts, such as higher-priced gifts, can be unlocked for the child 104. This incentive practice engages the child 104 in educational activities and helps in the learning process of the child. The occasion price threshold 206 allows users such as parents to set a purchase price threshold for a particular occasion. For example, a parent can set a $1000 limit for Christmas, and grandparents can set $250. Once the child has built his/her profile and has set up his/her preferences such as likes, dislikes, and interests in the profile, the recommendation engine 116 recommends relevant product options 210 based on the parameter preferences set by the child 104. The recommendation system 116 is coupled to the child's profile 200 and the product suppliers 209. For example, if the child 104 chooses Minnie Mouse®, Dora the Explorer®, dollhouses, tennis shoes, and the color red, the recommendation engine 116 searches through the supplier or vendor sites and product inventories that are partners of the system of the present invention for bidding for the selected items.

The product suppliers 209 can be companies such as Amazon®, Target®, Walmart® or any other private retailer or vendor that are part of the children shopping platform 100. Once a supplier 209 has accepted the membership into the children's shopping platform 100, the supplier 209 can submit the product inventory and pricing for the products in the children's shopping platform 100. The product supplier 209, with the help of the recommendation system 116, can provide a relevant item based on the generated customer/child profile 200.

As stated supra, the recommendation engine 116 also recommends a package or a ‘click pack’ of all items for a given special occasion as a single unit. The list of suppliers 209 that are a part of the system 100 bid for the package for sending or sourcing these ‘click packs’. The server or the recommendation engine 116 sources the requirements of the package to the suppliers 209 with the intention of having the suppliers 209 ‘bid’ on the rights to supply the ‘click packs’ to said user. Once the suppliers 209 receive the required information from the servers and/or recommendation engine 116, the suppliers 209 bid for the relevant products on the shopping platform 100, and allow the users to buy their products as desired.

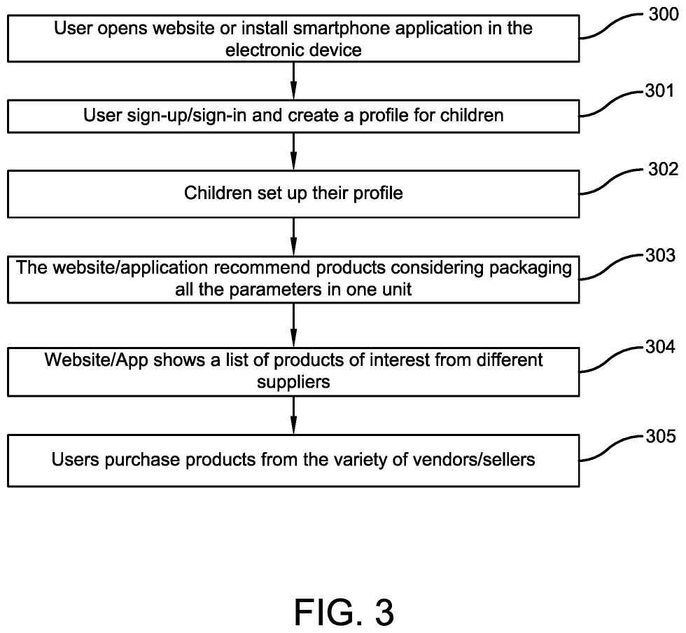

FIG. 3 illustrates a flow diagram showing exemplary steps of recommendation and purchase of products for children in the children's shopping platform of the present invention in accordance with the disclosed architecture. Initially, a user (a parent or a child) opens a shopping platform website or installs the shopping platform smartphone application in an electronic device such as a smartphone, laptop, mobile device or any other such type of device (Block 300). The website and the smartphone application provide user interfaces allowing the user to access the functionality offered by the system of the present invention. Then, the user signs-up and creates a profile on the website, or on the smartphone application interface. If the profile is already created, the user can sign-in and access the website or the smartphone application (Block 301). During the sign-up process, children can set up their profile using their names and general information which is encrypted and protected on a secure server. The general information can include first name, age, sex, and other such types of demographic information. Once the sign-up process is completed children are asked to provide answers to the questions from a preset database of questions. These questions are utilized to build a profile of the child using AI technology based on likes, dislikes, favorite movies, games, characters, gift types, clothes, colors, animals, interests, TV shows, etc. As the child clicks the answers to the questions, the shopping platform builds a profile based on interests, personality, likes and dislikes (Block 302). It should be noted that parents, relatives and friends of a child can also create their respective profiles in the system and can use the shopping platform for purchasing a gift for the child. Additionally, one user can invite other users to create a profile on the system and to purchase a gift for a child.

The recommendation engine based on the factors provided by the user recommends the personalized products (Block 303). Further, in Block 303, recommendation engine evaluates all the parameters or preferences of the user in one package and recommends the products to the user. Then, the website, or software application provides the list of products of the user's interest from the different suppliers on the display of the electronic device (Block 304). Users (including the child, parents, relatives or friends) can purchase the products from a variety of pre-determined vendors or sellers that sell the chosen or selected items within a predefined budget (Block 305).

The present invention allows a parent to put an upper spending limit or an occasion price threshold for gift items for a child. The limit can be different for different occasions. For example, the limit can be higher for a graduation than for a birthday to motivate the child to work hard in their studies. The limit can be applied by one or more users. For example, a parent can set a limit of $1,000 for Christmas, and grandparents can set $250. The children's shopping platform of the present invention provides products according to the profile of the child and pre-defined threshold.

It is to be appreciated that if the child picks all his/her Christmas gifts that he/she wants and the cost exceeds the limit, then an email can be automatically generated and sent to the parent regarding the price limit violation. The parent can then either choose to increase the occasion price threshold to purchase the entire thing or can invite a relative or a friend to purchase or contribute to the remaining items of the package chosen by the child.

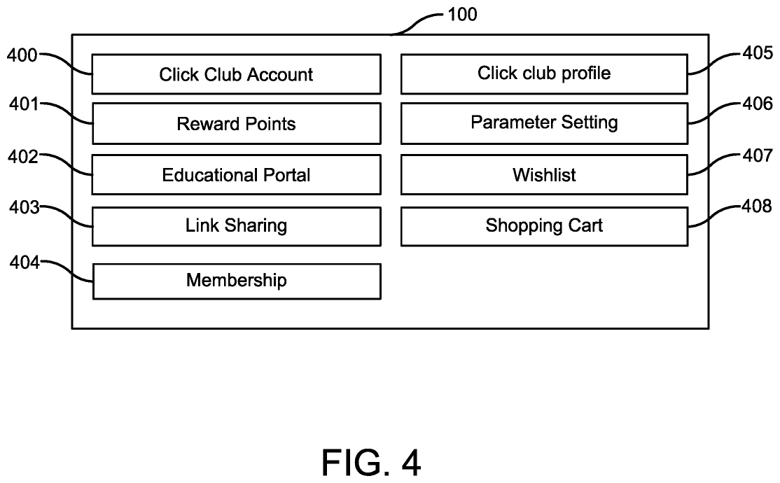

FIG. 4 illustrates a block diagram showing the different features of the children's shopping platform of the present invention in accordance with the disclosed architecture. The shopping and educational incentive platform 100 provides a plurality of features to enhance the shopping experience of the user. Users such as children, parents, grandparents, relatives, friends or others can access these features. The features of the shopping assistance platform 100 are user-friendly and provide a convenient and personalized shopping and gifting experience. The platform 100 can offer various customized and personalized discounts to the users on occasions such as birthdays, holidays and more, to provide an enhanced shopping experience.

The system 100 can provide users with a click club account feature 400 that enables users to store money in a “club” for upcoming events such as Christmas or for savings. The children/kids can also save money to purchase extra gifts throughout the year, or can purchase gifts for their siblings or friends at any time. The kid club account 400 is like a personalized digital wallet for purchasing gifts for oneself and others. The click club account feature 400 includes a way to transfer money from a user bank account, credit card, check card and via a one-time or recurring amount from the user's account to the click club account. The parent can add money into the click club account 400 throughout the year until the time of the occasion. The click club account feature 400 can also provide kids an opportunity to save their money into this account 400 and learn the process and importance of saving money for the future. The kids can then buy something they would like for themselves, a sibling or friend's birthday or for a special occasion.

The children's shopping platform 100 can offer a reward point feature 401. The reward point feature 401 can be built-up throughout the life of the account and reward points can be saved to buy prizes, gifts, etc. in the future. The reward feature 401 is similar to a loyalty program feature. For example, the system 100 can provide 1 point per $20 spent, or 10 points per $200 spent. Each point can be equivalent to a certain dollar amount value such as 1-point equivalent to $1.

The children's shopping platform 100 provides an education portal feature 402 for children. The children can access the education portal feature 402 without permission of the parent. Also, the parents can choose whether or not they want to engage themselves in the educational portal of the shopping platform 100. If the child chooses the education portal feature 402, he or she can be provided with the higher level and challenging learning activities such as math problems, spelling games, reading comprehension, science or anything that interests the child. These learning activities provide children with an option to unlock gifts, or increased value of gifts, that may not be otherwise attainable through normal methods of merely picking from the lists of likes or interests.

The children's shopping platform 100 also provides a link-sharing feature 403. The link-sharing feature 403 allows parents, guardians or relatives to share the link of the items with other users who would like to sign-up to the shopping platform 100 to purchase something for the kids. The other users can click on the link and buy items for the occasion. The shared link can have the information related to gifts that the child wants for his/her birthday or any other occasions. Once the child selects the gifts, those gift choices will be sent to the companies for bidding, and when the parents select the winning bid prices, the gifts will upload to the available profile, and others associated with the profile will be able to select gifts from the site. The link-sharing feature 403 enables others to only have access to the available gifts for purchase and will not have access to any personal information or the like.

The children's shopping platform 100 can provide a membership feature 404. The membership cost for the consumer can be free. The membership feature 404 can be subscription-based for the suppliers or vendors. The membership feature 404 can also charge a percentage of sales made from business attained through the children's shopping platform 100. The membership feature 404 can also provide a premium membership option to the users who wish to pay a monthly fee for a predetermined list of additional premium benefits. The premium benefits can include free shipping rates, double reward points, discounts or other such types of benefits.

The children's shopping platform 100 provides users with a click club profile feature 405 that enables the users to build their profile, based on the pre-determined factors such as likes, dislikes, interests, favorites and other such types of factors. The children's shopping platform 100 provides parents with a parameter-setting feature 406. The parameter setting feature 406 enables the parents to set the parameter, such as a price threshold parameter, or educational portion parameter for the item. The children's shopping platform 100 provides children with a wishlist feature 407 that allows the children to create a wish list of gifts based on their choices. The children's shopping platform 100 also provides the users with a shopping cart option 408 that allows the users to purchase a product from the children's shopping platform 100. Users can add all the items they want to purchase in the shopping cart 408 and can purchase all the items at one time by checking out and providing the payment using the payment gateway options on the shopping platform 100.

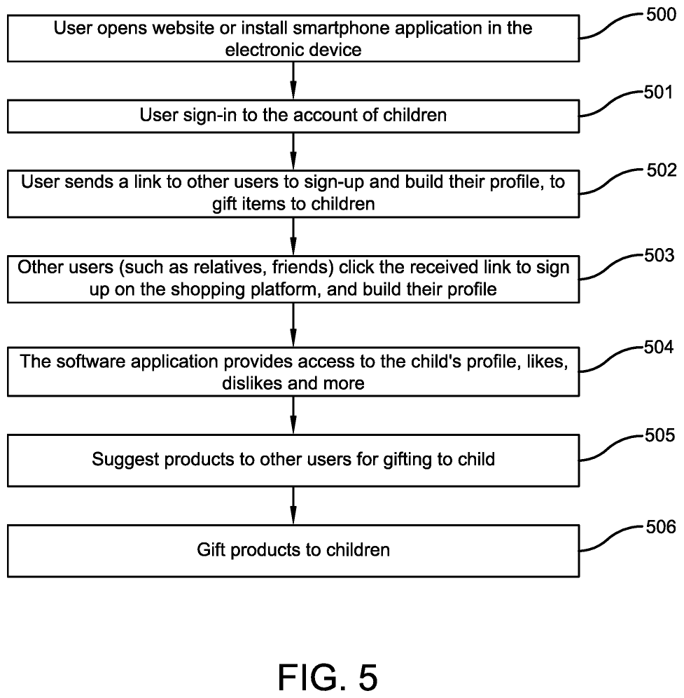

FIG. 5 illustrates a flow diagram showing the steps of sharing a link with other users using the children's shopping platform of the present invention in accordance with the disclosed architecture. As shown in FIG. 5, initially a user opens a website or installs the smartphone application in the electronic device such as a smartphone, laptop, mobile device or any other such type of device (Block 500). Then, the user signs-in to the children's account system using a username and a password (Block 501). After sign-in, the user can click on the gift items recommended by the recommendation engine based on the factors such as likes, dislikes, interests, etc. Then, the user can send a link to other users to sign-up and build their profile to gift items to the children (Block 502). Other users such as relatives and friends click the received link to sign-up on the children's shopping platform, and build their profile by providing the general information such as name, age, etc. (Block 503). Then, the children's shopping platform provides access to the child's profile, likes, dislikes, wish list and other such types of preferences to the other users (Block 504). The children's shopping platform suggests the products that are available for purchase for gifting to the child (Block 505). The other users can gift the selected products to children (Block 506).

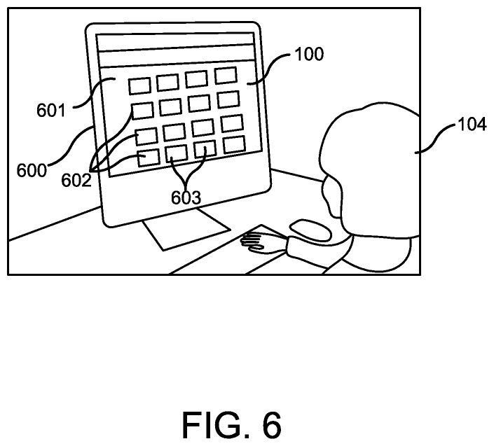

FIG. 6 illustrates a perspective view showing a child accessing the children's shopping platform interface on a portable device in accordance with the disclosed architecture. A user or child 104 can access the children's shopping platform 100 on an electronic device 600. The children shopping platform 100 interface is displayed on a display screen 601 of the electronic device 600. The children shopping platform 100 provides the child 104 an opportunity to engage in the shopping experience for special occasions, graduations, holidays, birthdays or any other such types of events. The children's shopping platform 100 also allows the child 104 to engage in educational level activities 602 such as spelling games, math problems, reading comprehension, science or anything that interests the child 104. The children's shopping platform 100 provides the user with product inventories 603 such as gifts, toys or other items based on the preferences or factors chosen by the user 104.

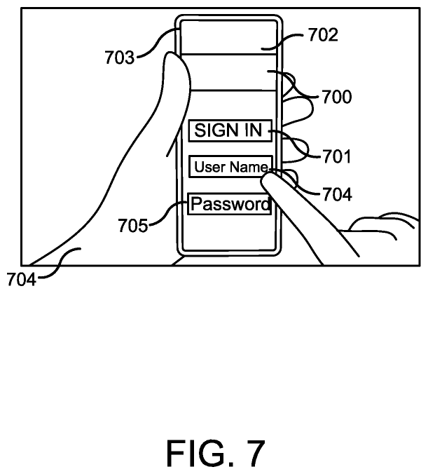

FIG. 7 illustrates a perspective view showing a sign-in interface of the software application of the children's shopping platform of the present invention in accordance with the disclosed architecture. The children's shopping platform software application 700 provides a user 104 with a sign-in interface 701. The sign-in interface 701 is displayed on the display screen 702 of the electronic device 703 with the installed children's shopping platform software application 700. The user 104 can provide a username 704 and a password 705 on the sign-in interface 701. Once the sign-in process is complete, the user 104 can access the content such as, product inventories, educational programs and other content of the children's shopping platform 100.

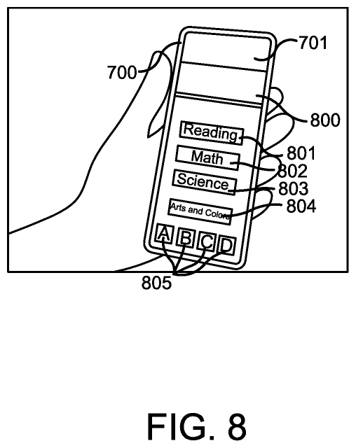

FIG. 8 illustrates a perspective view showing different interests displayed by the software application of the present invention in accordance with the disclosed architecture. As shown in FIG. 8, the children's shopping platform 100 provides users with various interests, school subjects, characters, movies, books, etc. that can be displayed on a display screen 702 of the electronic device 703. The shopping platform 100 can provide users with one or more interest options such as Reading 801, Math 802, Science 803, Arts and colors 804 and any other such types of interest options. The shopping platform 100 can provide users with an alphabetical listing 805 of all available options which will enhance the user's search experience.

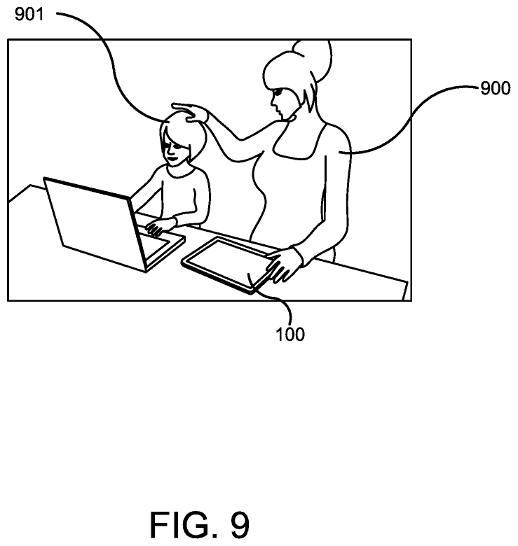

FIG. 9 illustrates a perspective view of a parent and a child shopping together using the shopping and educational platform of the present invention in accordance with the disclosed architecture. As shown, the children's shopping platform 100 allows a parent 900 and a child 901 to shop together for special occasions, graduations, holidays, birthdays or any other event that may warrant gift giving. The children's shopping platform 100 allows parents 900 to input the price amount and other parameters for the types of items the child 901 may select. The child 901 can compete against the tasks or parameters set by the parents and can select the gift item based on the likes, dislikes, or interests.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “Children's shopping combined with educational program system”, “children's shopping platform system”, “shopping and educational incentive platform”, “children's shopping platform”, “shopping platform” and “system”, are interchangeable and refer to the children's shopping platform system 100 of the present invention.

Notwithstanding the forgoing, the children's shopping platform system 100 of the present invention can include any additional component to enhance the functionality and efficiency of the children's shopping platform system 100 and the system 300. One of ordinary skill in the art will appreciate that the configuration, components of the children shopping platform system 100 as shown in the FIGS. are for illustrative purposes only, and that many other configurations and components are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A computer implemented method of allowing a user to engage in an electronic shopping experience, the method comprising the steps of:
creating a child profile and a parent profile, wherein said child profile is based on at least an age, a gender, and a preference of a child, and further wherein said parent profile includes at least a first control;
receiving a selection of gift items from said child;
selecting a plurality of said gift items for purchase from said selection of gift items;
packaging said plurality of said gift items into a click pack unit;
sending said click pack unit to a plurality of registered suppliers for a bid;
receiving one or more bids from one or more of said plurality of registered suppliers to supply said click pack unit;
selecting one supplier for supplying said click pack unit;
comparing said bid of said one supplier to said at least a first control; and
sharing a link to said click pack unit to an associated user.

2. The method of claim 1, wherein said preference is selected from a group consisting of a like, a dislike, an interest, a personality, an educational interest, a color, an animal, and a toy.

3. The method of claim 1, wherein said preference is selected from a group consisting of a favorite movie, a favorite game, a hobby, a product preference, and a store preference.

4. The method of claim 1, wherein said receiving a selection of gift items from said child is for a specific occasion.

5. The method of claim 1, wherein said at least first control includes a cost threshold limit for said click pack unit set by said parent.

6. The method of claim 5, wherein said cost threshold limit is a predetermined ceiling limit on the cost of said click pack unit based on a specific occasion.

7. The method of claim 6 further comprising a step of providing an educational program for completion by said child in order to increase said predetermined ceiling limit based upon a progress of said educational program.

8. The method of claim 5 further comprising a step of requesting an increase to said predetermined ceiling limit when said cost of said click pack unit exceeds said predetermined ceiling limit.

9. The method of claim 1, wherein said sharing a link to said click pack unit to the associated user includes an invite to purchase an item from said click pack unit.

10. The method of claim 6, wherein said sharing a link to said click pack unit to the associated user includes an invite to purchase an item from said click pack unit.

11. The method of claim 10 further comprising a step of issuing another invite to said associated user to purchase another item from said click pack unit when said cost of said click pack unit exceeds said predetermined ceiling limit.

12. A computer implemented method of allowing a user to engage in an electronic shopping experience, the method comprising the steps of:
creating a child profile and a parent profile, wherein said child profile is based on at least an age, a gender, and a preference of a child, and further wherein said parent profile includes at least a first control;
receiving a selection of gift items from said child;
selecting a plurality of said gift items for purchase from said selection of gift items;
packaging said plurality of said gift items into a click pack unit, wherein said at least first control includes a predetermined cost threshold limit for said click pack unit set by a parent of the child; and
providing educational programs for completion by said child in order to increase said predetermined cost threshold limit based upon a progress of said educational programs.

13. The method of claim 12, wherein said preference is selected from a group consisting of a like, a dislike, an interest, a personality, an educational interest, a color, an animal, and a toy.

14. The method of claim 12, wherein said preference is selected from a group consisting of a favorite movie, a favorite game, a hobby, a product preference, and a store preference.

15. The method of claim 12 further comprising:
sending said click pack unit to a plurality of registered suppliers for a bid;
receiving one or more bids from one or more said plurality of registered suppliers to supply said click pack unit;
selecting one supplier for supplying said click pack unit; and
comparing said bid of said one supplier to said predetermined cost threshold limit.

16. The method of claim 15 further comprising a step of requesting an increase to said predetermined cost threshold limit when said cost of said click pack unit exceeds said predetermined cost threshold limit.

17. The method of claim 16 further comprising a step of sharing a link to said click pack unit to an associated user.

18. The method of claim 17, wherein said sharing a link to said click pack unit to the associated user includes issuing an invite to purchase an item from said click pack unit.

19. The method of claim 18 further comprising a step of issuing another invite to the associated user to purchase another item from said click pack unit when said cost of said click pack unit exceeds said predetermined cost threshold limit.

20. A computer implemented method of allowing a child, a parent of the child, and an associated user to engage in an electronic shopping experience, the method comprising the steps of:
creating a child profile and a parent profile, wherein said child profile is based on at least an age, a gender, and a preference of the child, and further wherein said parent profile includes at least a first control;
receiving a selection of gift items from said child;
selecting a plurality of said gift items for purchase from said selection of gift items;
packaging said plurality of said gift items into a click pack unit, wherein said at least first control includes a cost threshold limit for said click pack unit set by said parent;
providing educational programs for completion by said child in order to increase said cost threshold limit based upon a progress of said educational programs, wherein said preference is selected from a group consisting of a like, a dislike, an interest, a personality, an educational interest, a favorite movie, a favorite game, a hobby, a product preference, and a store preference;
using artificial intelligence to analyze said preference of said child profile; and
recommending a plurality of gift items to the associated user.
